(19) United States
(12) Patent Application Publication
(10) Pub. No.: US 2017/0368336 A1
(43) Pub. Date: Dec. 28, 2017

(54) DEVICE, METHODS AND REPLACEABLE RESERVOIR DESIGN FOR TRANSDERMAL DRUG DELIVERY BY CONTACTLESS ELECTROPORATION, IONTOPHORESIS AND NANONIZATION

(71) Applicants: DAHSHIARN CHIAO, Naperville, IL (US); CHIEN-AN CHANG, Taoyuan (TW)

(72) Inventors: DAHSHIARN CHIAO, Naperville, IL (US); CHIEN-AN CHANG, Taoyuan (TW)

(73) Assignee: DAHSHIARN CHIAO

(21) Appl. No.: 15/189,001

(22) Filed: Jun. 22, 2016

(51) Int. Cl. A61N 1/10; A61K 9/00

(52) U.S. Cl. CPC A61N 1/10; A61K 9/0009

(57) ABSTRACT

The present invention provides a contactless technique for delivering a charged drug into a body tissue using a replaceable reservoir or cartridge loaded with the charged drug and applying to it an electrical gradient, a high voltage pulses and an electrostatic force, such that the nanonized charged drug moves into the tissue under the influence of these electrical field and force.

### CROSS REFERENCE TO RELATED APPLICATION

### FIELD OF THE INVENTION

The present invention provides a contactless technique for delivering a charged drug into a body tissue using a replaceable reservoir or cartridge loaded with the charged drug and applying to it an electrical gradient, a high voltage pulses and an electrostatic force, such that the nanonized charged drug moves into the tissue under the influence of these electrical field and force.

### BACKGROUND OF THE INVENTION

The disclosed drug applicator comprises at least one nozzle for contactless electrostatically charged administration, an associated replaceable reservoir or cartridge for storing at least one charged drug, a special arranged grounding configuration for conducting away cumulated electrostatic field away from the administration nozzles region and target tissue, and a nozzle-ring configuration for generating the potential gradient electric field in the vicinity of the nozzles to such an extent that administration of charged drugs is focused when the forward extremity of the nozzle-ring configuration is brought within a predetermine distance from an earthed target to be sprayed. The drug applicator has a replaceable reservoir or cartridge portion, and the applicator has apply an electrical gradient, a high voltage pulses and an electrostatic force through a contactless and inconsecutive administration of charged drug particle, such that the nanonized charged drug moves into the tissue under the influence of these electrical field and force.

The drug applicator is designed to be a portable, hand-held, self-contained, battery or adaptor operated charged drug releasing, with a replaceable reservoirs or cartridges system. Additional embodiments of the present invention comprise nozzle-ring configuration and methods for effectively and efficiently releasing drug, personal care and personal hygiene products stored in replaceable reservoirs or cartridges system.

### SUMMARY OF THE INVENTION

The limitations of the prior art are overcome in embodiments of the present invention. A first embodiment of the present invention comprises a replaceable reservoirs or cartridges system which having at least one replaceable reservoir or cartridge. Each replaceable reservoir or cartridge further comprises an electrostatically charged administrable material storage region, a replaceable or fixed spraying nozzles region and a nozzle-ring configuration. The material storage region comprises a material storage configuration and a compressible configuration. The replaceable spraying nozzles region comprises material conducting channels, a high voltage conducting configuration and various arranged nozzles apparatus. The nozzle-ring configuration comprises a conducting or semi-insulating material which surrounds the nozzles and yields the necessary electric field around the nozzles during the initial application of voltage for spraying of the material. In general where the material to be sprayed is in the form of a liquid, the high Voltage circuitry will have the effect of causing the propulsion of one or more filaments or ligaments of liquid from the nozzle, which ligament(s) breakup into electrostatically charged droplets. Typically the material applied will comprise drug or cosmetic nature such as personal care products, e.g. deodorants, antiperspirants, anti-bacterials, perfumes, hair sprays, fresheners, moisturizers and conditioners etc.

A second embodiment of the present invention comprises a controllable material conducting gate configuration. The controllable material conducting gate configuration typically constructed with a film or membrane is mounted in fixed relation to both the spraying nozzles region and material storage region in an airtight fashion, and material in electrostatically charged administrable form may be released from the material storage region to the spraying nozzles region when the material conducting gate region is opened. The controllable material conducting gate region may however be opened through a penetrating action, usually in a way of stabbing or screwing a pin or rod through the material conducting gate region, in adjustable relation to the axis of the top of the replaceable reservoirs or cartridges. With a design of the controllable material conducting gate configuration, a replaceable electrostatically charged administrable material reservoir or cartridge can prevent further precipitation or clogging of the administrable material within the nozzles during shipping or storage. In one variant of the third embodiment, the controllable material conducting gate or seal configuration typically constructed with a film or membrane is mounted in fixed relation to the material storage region in a sealed fashion, and material in electrostatically charged administrable form may be released from the material storage region to the spraying nozzles region after both the material conducing gate region is opened or removed (e.g. by removing seal, penetrating seal, etc.) and the replaceable spraying nozzles region is attached to the material storage region.

A third embodiment of the present invention comprises a specific structure for a nozzle-ring configuration. The specific structure comprises, a physical configuration mounted in fixed relation to the body of the device and the ring configuration is in the form of an annular cable mounted on the body of device, and usually in substantially concentric relation with the spraying nozzles unit. The ring configuration and the spraying nozzles unit may however be adjustable with respect to one another in the direction of spraying. Where the nozzles and/or the ring configuration is adjustable, preferably the limits of adjustment are such that the nozzle-ring configuration, over substantially its full range of adjustment, has its forward extremity located forwardly of the nozzles. The arrangement is conveniently such that, in all positions of relative adjustment, spraying is focused when the forward extremity of the nozzle-ring configuration is within a certain distance from an earthed target. In addition, the ring configuration surrounds the nozzles and yields the necessary electric field around the nozzles during the initial application of voltage for spraying of the material thus spraying is focused and also generating an electrical gradient, iontophoresis or electroporation effect to enhance sprayed material transport through the skin until the forward extremity of the nozzle-ring configuration is within a certain distance from a human skin target or an earthed target. In an alternative embodiment, the nozzle-ring configuration may be composed of a semi-insulating material which is coupled to a source of high voltage forming part of the device and has sufficient conductivity to permit a potential to be established at a location forwardly of the nozzles which is of the same polarity as that applied to the material emerging at the nozzle.

A fourth embodiment of the present invention comprises a hand-held drug applicator which having at least one replaceable reservoir or cartridge, at least one replaceable spraying nozzles configuration, a ring configuration, a grounding configuration, a high voltage generator, a power source, an external grounding wire configuration, a control circuit, a grounding circuit design, a trigger. The special arranged grounding configuration is directly connected with an external grounding wire configuration through the grounding circuit design. Through the special arranged grounding configuration the cumulated electrostatic field generating by the high voltage circuit forms in the vicinity of the spraying nozzles region is conducted away from the spraying nozzles region and the sprayed target. In one variant of the fourth embodiment, the special arranged grounding configuration is directly connected with an external grounding wire configuration and a special AC to DC adaptor through the grounding circuit design. Through the special arranged grounding configuration the cumulated electrostatic field generating by the high voltage circuit forms in the vicinity of the spraying nozzles region is conducted away from the spraying nozzles region and the sprayed target, and then released into a ground or an AC power source through the special AC to DC adaptor. The grounding circuit design of the special AC to DC adaptor is composed of a capacitor (e.g. Y or X type capacitor) which is directly coupling the secondary core to the primary core of a power transformer within a double blade (AC plug) power adaptor (AC to DC adaptor) so that, in use, the cumulated electrostatic charge/field issuing from the spraying nozzles in a direction away from said spraying nozzles is conducted away from the spraying nozzles region and the sprayed target through the capacitor, secondary core of a power transformer, and primary core of a power transformer then into the grounding of a AC jack or a power source. In yet another variant of the fourth embodiment, a special arranged shielding configuration is directly connected with an external grounding wire configuration and a special AC to DC adaptor through the grounding circuit design. Through the special arranged shielding configuration the generated magnetic wave is conducted into the ground, said spray having a high voltage and a polarity and wherein a magnetic wave forms in the vicinity of the nozzles during use.

In one variant of the fourth embodiment, the special adjustable arranged grounding configuration is directly connected with an external grounding wire configuration and a special DC or AC power source through the grounding circuit design. Through the special arranged grounding configuration the cumulated electrostatic field generating by the high voltage circuit forms on the sprayed target is conducted away from on the sprayed target, and then released into a ground or a special DC or AC power source through the special adjustable grounding circuit design in a inconsecutively fashion and thus generate a high voltage pulses.

In yet another variant of the fourth embodiment, the pulse control high voltage generator can generate pulsed electrostatic field or high voltage when coordinated with special adjustable grounding circuit design.

### BRIEF DESCRIPTION OF THE FIGURES

The above and other objects and advantages of this invention will be apparent upon consideration of the following detailed description, taken in conjunction with the accompanying drawings in which like characters refer to like elements throughout and in which:

FIG. 1 depicts front, rear and perspective views of a hand-held drug applicator and replaceable reservoirs or cartridge made in accordance with a preferred embodiment of the present invention;

FIG. 2 depicts top and cross-sectional views of a replaceable reservoir or cartridge made in accordance with a preferred embodiment of the present invention;

FIG. 2A depicts top and cross-sectional views of alternate replaceable reservoir or cartridge made in accordance with a preferred embodiment of the present invention;

FIG. 2B depicts top and cross-sectional views of alternate replaceable reservoir or cartridge made in accordance with a preferred embodiment of the present invention;

FIG. 2C depicts top and cross-sectional views of alternate replaceable reservoir or cartridge made in accordance with a preferred embodiment of the present invention;

FIG. 3 depicts front, top, and perspective views of a hand-held drug applicator and replaceable reservoirs or cartridge made in accordance with a preferred embodiment of the present invention;

FIG. 4 depicts top and cross-sectional views of a replaceable reservoir or cartridge made in accordance with a preferred embodiment of the present invention;

FIG. 4A depicts top and cross-sectional views of alternate replaceable reservoir or cartridge made in accordance with a preferred embodiment of the present invention;

FIG. 5 depicts top and cross-sectional views of a replaceable reservoir or cartridge made in accordance with a preferred embodiment of the present invention;

FIG. 6 depict front, rear and cross-sectional view of a hand-held drug applicator having at least one replaceable reservoir or cartridge, at least one replaceable spraying nozzles region, a ring configuration, a high voltage generator, a power source, control circuit, trigger and terminals connected to a high voltage generator made in accordance with a preferred embodiment of the present invention;

FIG. 7 depict front, rear and cross-sectional view of a hand-held drug applicator having at least one replaceable reservoir or cartridge, at least one replaceable spraying nozzles region, a ring configuration, a grounding configuration, an external grounding wire configuration, a high voltage generator, a power source, control circuit, trigger and terminals connected to a high voltage generator made in accordance with a preferred embodiment of the present invention;

FIG. 8 depict front, rear and cross-sectional view of a hand-held drug applicator having at least one replaceable reservoir or cartridge, at least one replaceable spraying nozzles region, a ring configuration, a grounding configuration, an external grounding wire configuration, a high voltage generator, a power source, control circuit, trigger, adjustable grounding circuit design and terminals connected to a high voltage generator made in accordance with a preferred embodiment of the present invention;

FIG. 9 depict front, rear and cross-sectional view of an alternate hand-held drug applicator having at least one replaceable reservoir or cartridge, at least one replaceable spraying nozzles region, a ring configuration, a grounding configuration, a special AC to DC adaptor, an external grounding wire configuration, a high voltage generator, a power source, control circuit, trigger and terminals connected to a high voltage generator made in accordance with a preferred embodiment of the present invention;

FIG. 10 depict front, rear and cross-sectional view of a hand-held drug applicator having at least one replaceable reservoir or cartridge, at least one ring configuration, a grounding configuration, an external grounding wire configuration, and a trigger made in accordance with a preferred embodiment of the present invention;

FIG. 11 depict front, rear and cross-sectional view of an alternate hand-held drug applicator having at least one replaceable reservoir or cartridge, at least one replaceable spraying nozzles region, a ring configuration, a shielding configuration, an external grounding wire configuration, a high voltage generator, a power source, control circuit, trigger and terminals connected to a high voltage generator made in accordance with a preferred embodiment of the present invention;

FIG. 12 depict front, rear and cross-sectional view of an alternate hand-held drug applicator having at least one replaceable reservoir or cartridge, at least one replaceable spraying nozzles region, a ring configuration, a shielding configuration, an external grounding wire configuration, a high voltage generator, a power source, control circuit, trigger, adjustable grounding circuit design and terminals connected to a high voltage generator made in accordance with a preferred embodiment of the present invention;

FIG. 13 depict front, rear and cross-sectional view of an alternate hand-held drug applicator having at least one replaceable reservoir or cartridge, at least one replaceable spraying nozzles region, a ring configuration, a shielding configuration, a special AC to DC adaptor, an external grounding wire configuration, a high voltage generator, a power source, control circuit, trigger and terminals connected to a high voltage generator made in accordance with a preferred embodiment of the present invention; and

FIG. 14 depict front, rear and cross-sectional view of an alternate hand-held drug applicator having at least one replaceable reservoir or cartridge, at least one ring configuration, a shielding configuration, an external grounding wire configuration, and a trigger made in accordance with a preferred embodiment of the present invention.

### DETAILED DESCRIPTION OF THE PREFERRED EMBODIMENTS

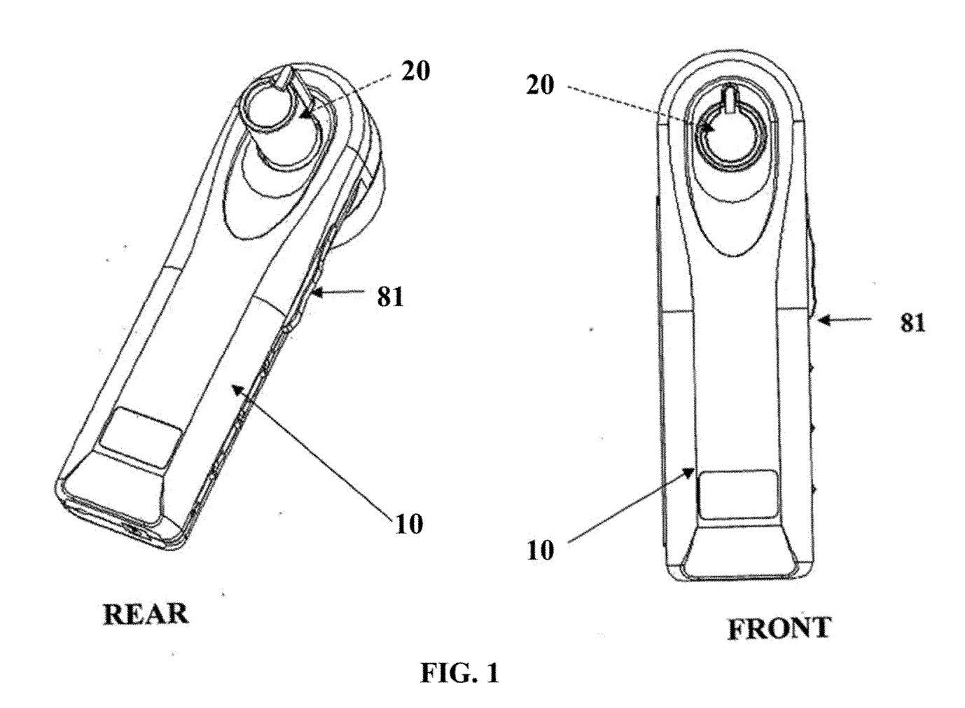

A hand-held drug applicator made in accordance with the first preferred embodiments of the present invention is depicted in perspective, front and rear views in FIG. 1. The hand-held drug applicator 10 integrated replaceable reserve or cartridge 20 is depicted attached to a hand-held drug applicator 10.

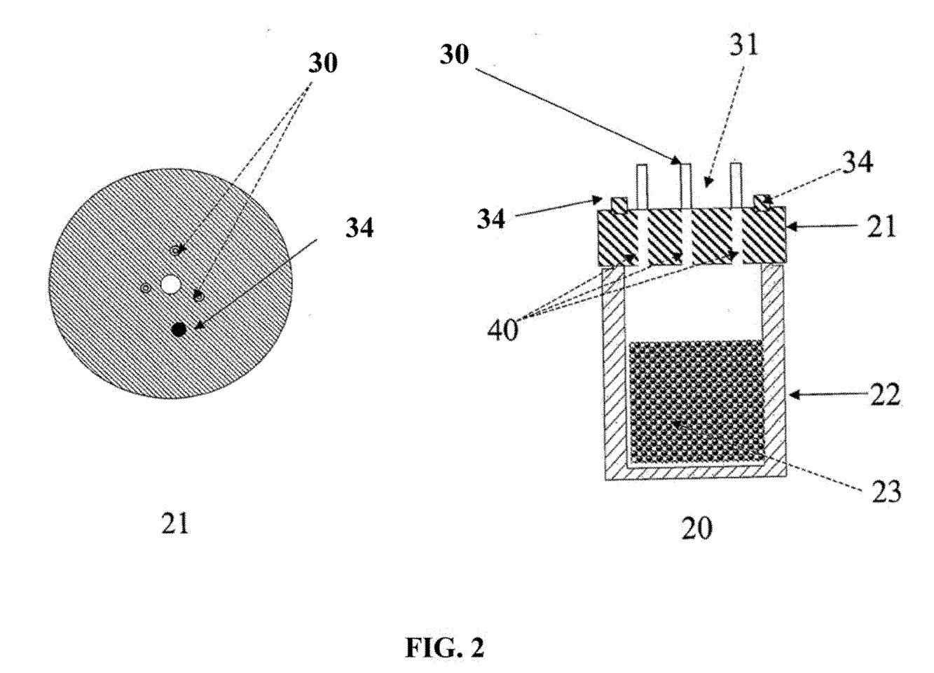

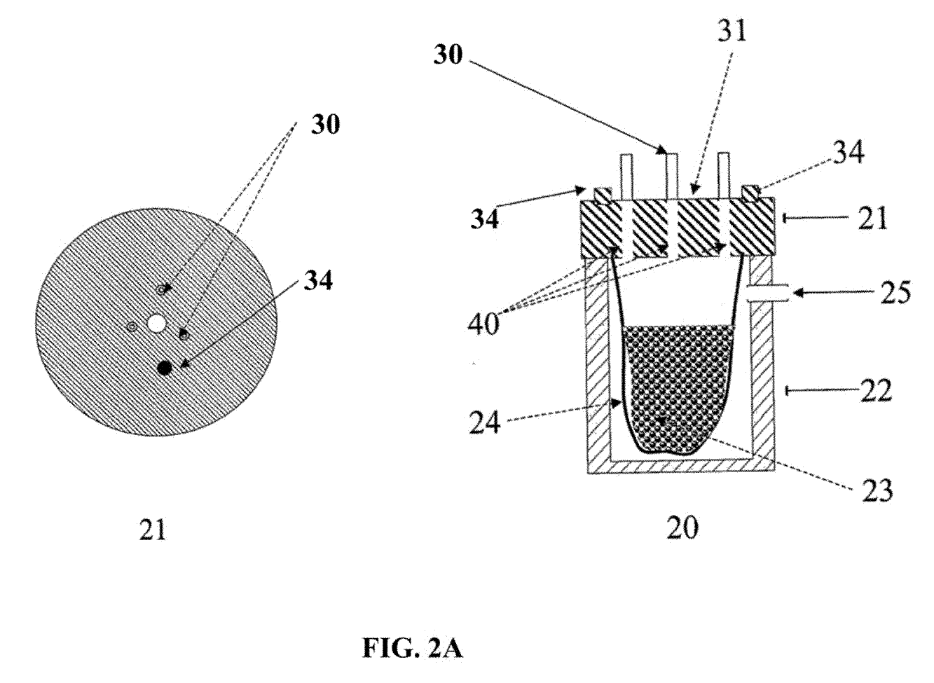

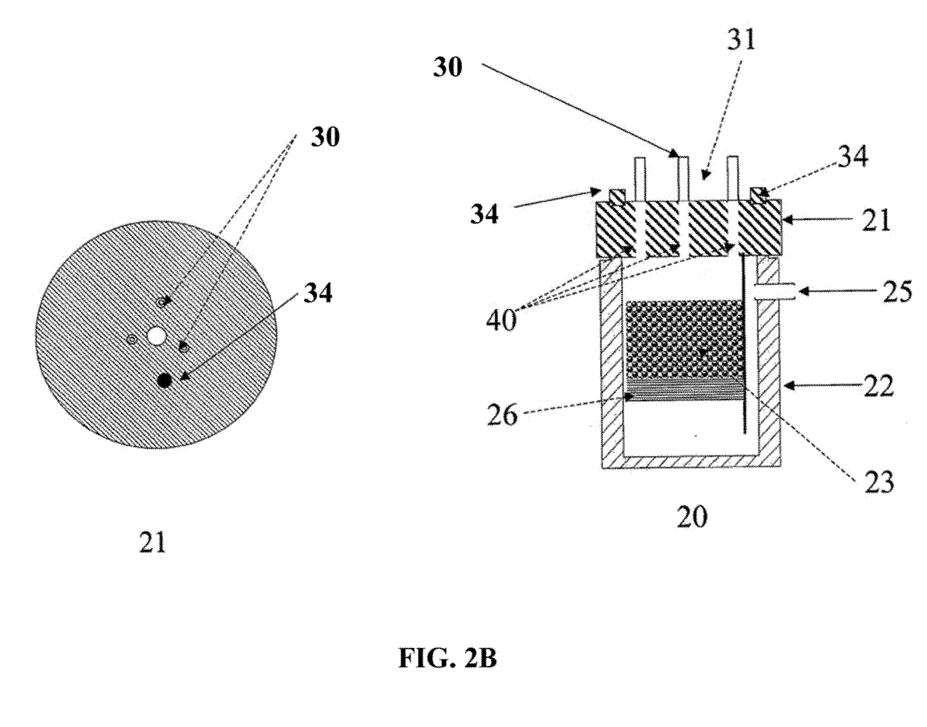

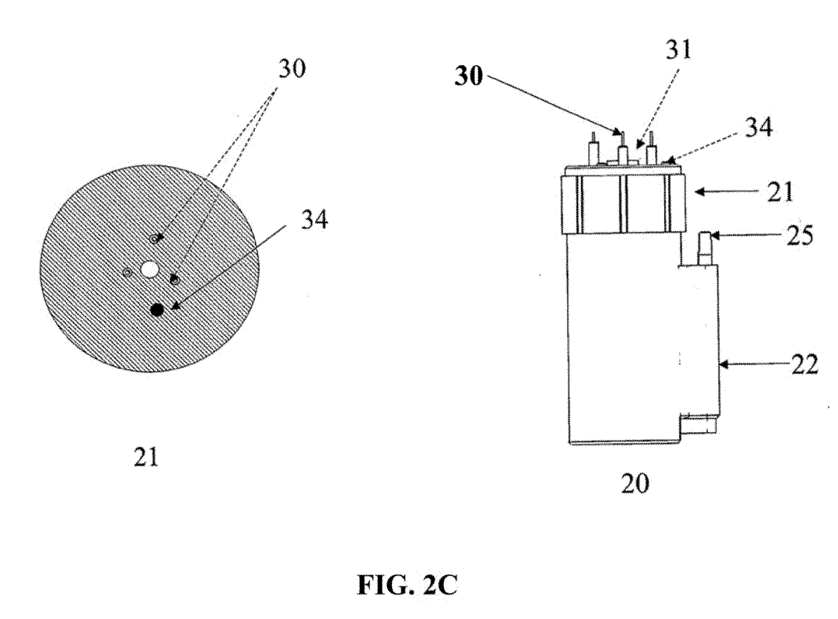

A replaceable reservoir or cartridge made in accordance with the first preferred embodiments of the present invention is depicted in top and cross-sectional views in FIG. 2. The replaceable reservoir or cartridge 20 shown in FIG. 2 comprises an electrostatically charged administrable material storage region 22, each of which is contained in a three-dimensional sector of the replaceable reservoir or cartridge 20. The replaceable reservoir or cartridge 20 has a material conducting tube region 40 that is continuously in directly connect with the spraying nozzles region 31. Delivery of an electric charge through a selected terminal 34 to a designated spraying nozzles 30 sublimates the electrostatically charged administrable material into many droplets which are focused when the forward extremity of the nozzle-ring configuration 51 is brought within a predetermine distance from an earthed target to be sprayed.

In one alternative embodiment shown in FIG. 2A, a replaceable reservoir or cartridge 20 comprises an electrostatically charged administrable material storage region 22, which is contained in a three-dimensional sector of the replaceable reservoir or cartridge 20. The replaceable reservoir or cartridge 20 has a material conducting tube region 40 and an air conduct channel configuration 25 that is continuously in directly connect with the pumping air conduct channel configuration 71 in which air pressure from the pumping means 70 is produced in response to operation of actuating means by the user. The membrane 24 installed in a relation within the replaceable reservoir 20 is compressed when the air pressure is applied. The compressed membrane 24 then push electrostatically charged administrable material 23 through the material conducting tube region 40 to the spraying nozzles region 31.

FIG. 2B depicts variants of the second preferred embodiment in which the piston configuration 26 is installed in a relation within the replaceable reservoir 20.

FIG. 2C depicts variants of the second preferred embodiment in which the air conduct channel 25 is constructed in a relation to the replaceable reservoir 20.

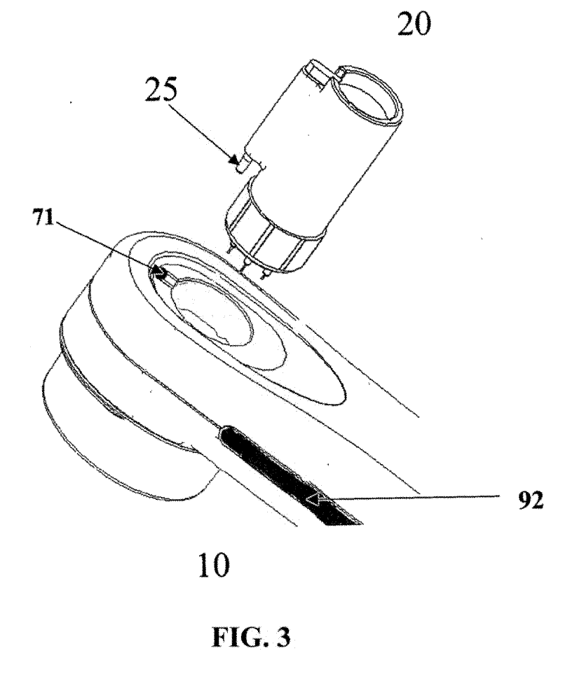

A hand-held drug applicator made in accordance with the second preferred embodiments of the present invention is depicted in perspective, top and front views in FIG. 3. The hand-held drug applicator 10 integrated replaceable reservoirs or cartridges 20 is depicted attaching to a hand-held drug applicator 10.

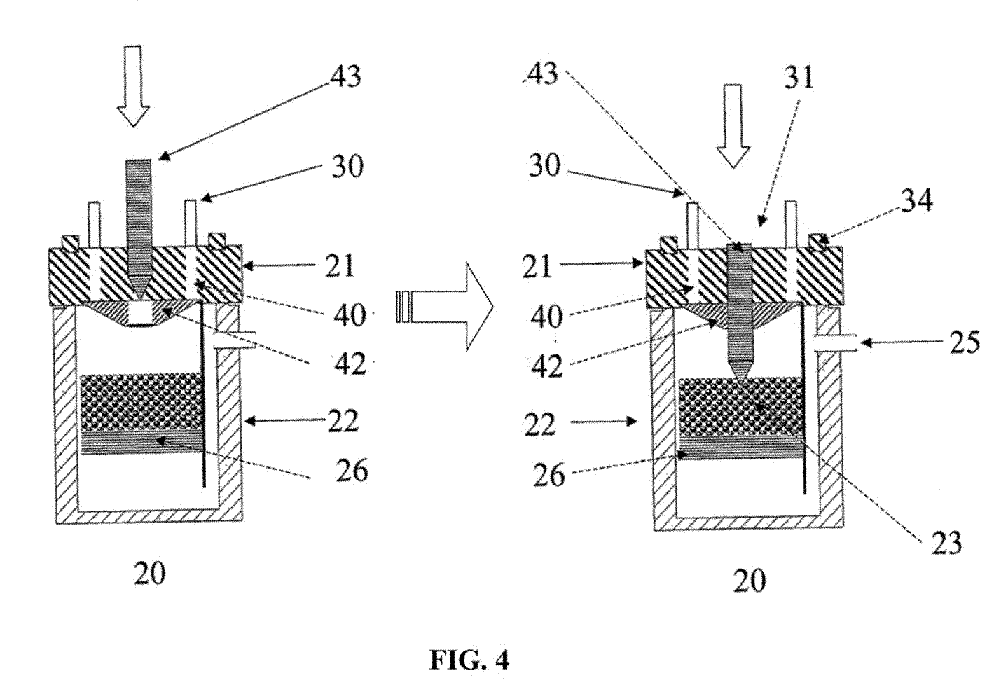

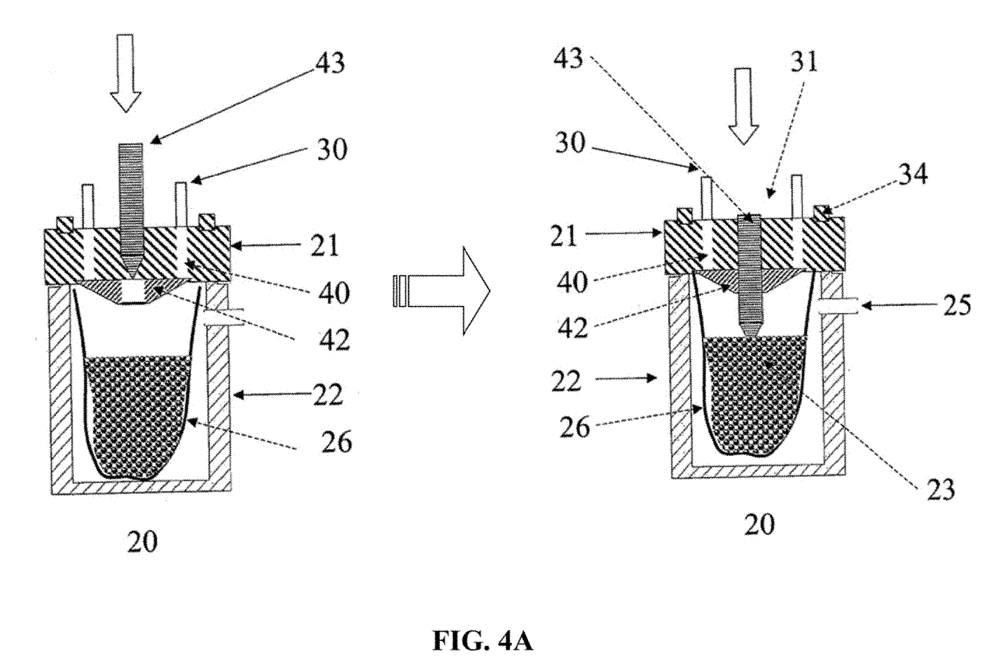

In another alternative embodiment of the present invention shown in FIGS. 4 and 4A, a replaceable reservoir or cartridge 20 comprises a controllable material conducting gate region 42, which is contained in a three-dimensional sector of the replaceable reservoir or cartridge. The replaceable reservoir or cartridge 20 has a material conducting tube region 40 and a controllable material conducting gate region 42 that is closed and block the connection between the material conducting tube region 40 and the electrostatically charged administrable material storage region 22. The gate configuration 42 installed in a relation within the replaceable reservoir 20 is opened when the pin 43 is applied to penetrate the gate region 42. Electrostatically charged administrable material 23 released from the material storage region 22 through the material conducting tube region 40 to the spraying nozzles region 31.

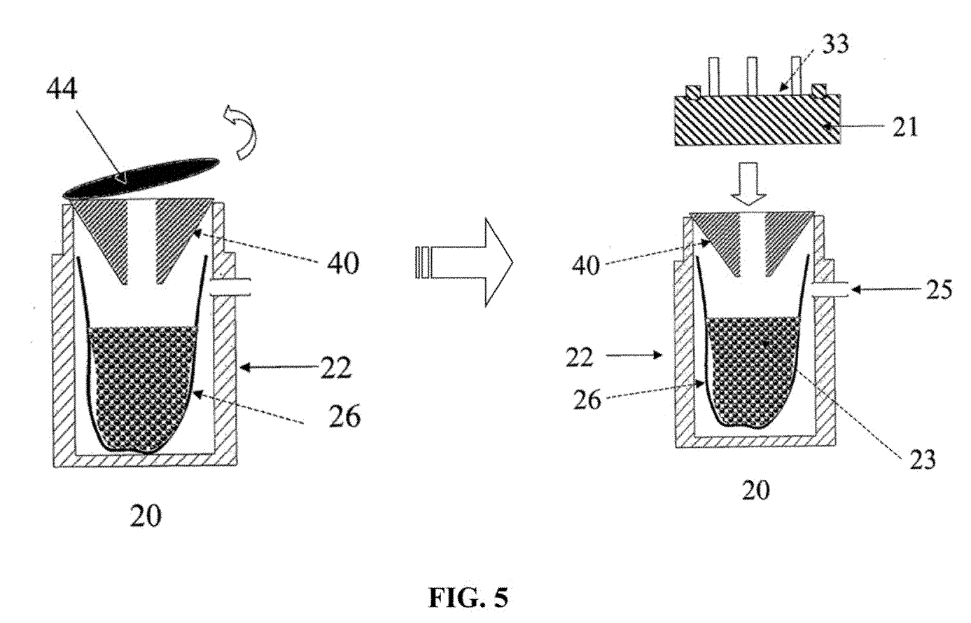

In yet other embodiment of the present invention shown in FIG. 5, a replaceable reservoir or cartridge 20 comprises a replaceable spraying nozzles configuration 33, which is installed in a relation to the top of the replaceable reservoir or cartridge. The replaceable reservoir or cartridge 20 has a replaceable spraying nozzles region 33 and a removable sealing configuration 44 that is sealed and block the connection between the material conducting tube region 40 and the electrostatically charged administrable material storage region 22. When the removable sealing configuration 44 installed in a relation within the replaceable reservoir 20 is opened or removed the electrostatically charged administrable material 23 released from the material storage region 22 through the material conducting tube region 40 to the spraying nozzles region 31.

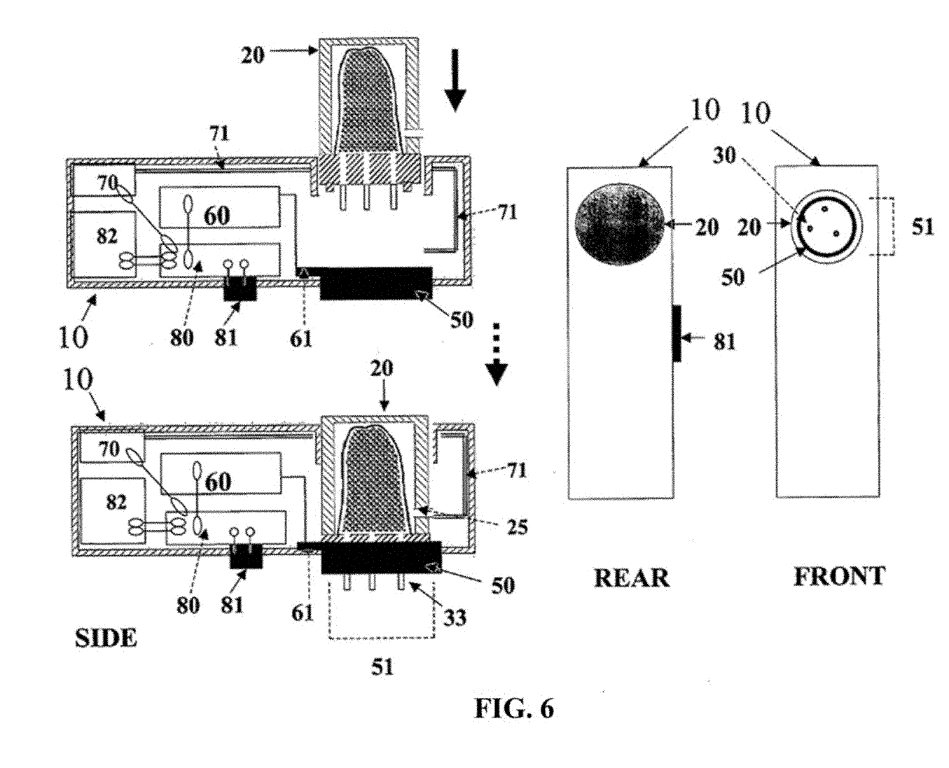

A complete hand-held drug applicator 10 having replaceable reservoir or cartridge 20, at least one replaceable spraying nozzles configuration 33, a ring configuration 50, a high voltage generator 60, a power source 82, control circuit 80, a trigger 81, a pumping means 70 and terminals 61 connected to the high voltage generator 60 made in accordance with the first preferred embodiment of the present invention is shown in FIG. 6. The replaceable reservoir or cartridge 20 shown in FIG. 6 comprises an administrable material storage region 22, which is contained in a three-dimensional sector of the replaceable reservoir or cartridge. The hand-held drug applicator 10 has terminals 61 that directly connect with a high voltage generator 60 and the ring configuration 50. By connecting the ring configuration 50 to a designated terminal 34 from a replaceable reservoir or cartridge 20, delivery of an electric charge through ring configuration 50 and 34 sublimates the electrostatically charged administrable material into many droplets which are focused when the forward extremity of the nozzle-ring configuration 50 is brought within a predetermine distance from an earthed target to be sprayed.

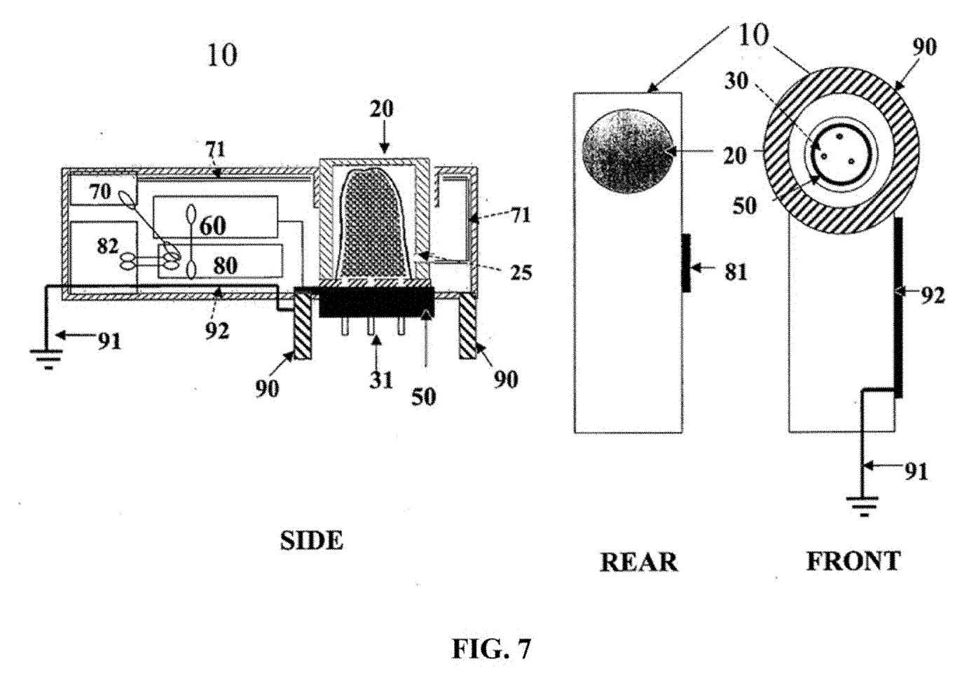

A complete hand-held drug applicator 10 having replaceable reservoir or cartridge 20, at least one replaceable spraying nozzles configuration 33, a ring configuration 50, a grounding configuration 90, a high voltage generator 60, a power source 82, an external grounding wire configuration 91, control circuit 80, a grounding circuit design 92, a trigger 81, a pumping means 70 and terminals 61 connected to the high voltage generator 60 made in accordance with the fourth preferred embodiment of the present invention is shown in FIG. 7. The replaceable reservoir or cartridge 20 shown in FIG. 7 comprises an administrable material storage region 22, which is contained in a three-dimensional sector of the replaceable reservoir or cartridge 20. The hand-held ding applicator 10 has a grounding configuration 90 that directly connect with an external grounding wire configuration 91 through the grounding circuit design 92. The cumulated electrostatic field generating by the high voltage circuit forms in the vicinity of the spraying nozzles region 31 is conducted away from the spraying nozzles region 31.

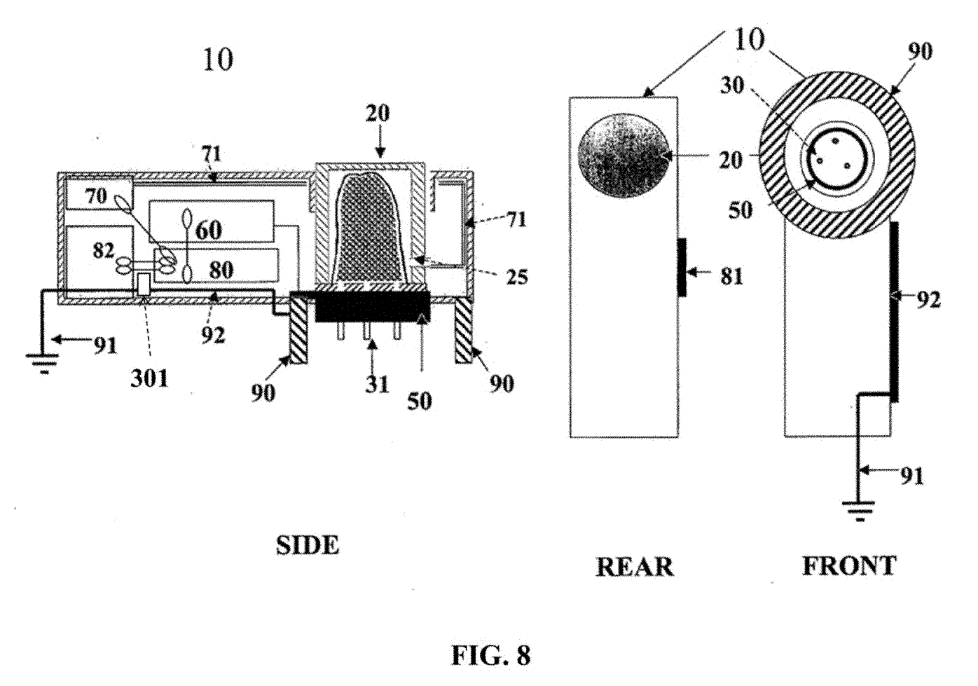

A complete hand-held drug applicator 10 having replaceable reservoir or cartridge 20, at least one replaceable spraying nozzles configuration 33, a ring configuration 50, a grounding configuration 90, a high voltage generator 60, a power source 82, an external grounding wire configuration 91, control circuit 80, a grounding circuit design 92, a trigger 81, a pumping means 70 and terminals 61 connected to the high voltage generator 60 made in accordance with the fourth preferred embodiment of the present invention is shown in FIG. 8. The replaceable reservoir or cartridge 20 shown in FIG. 8 comprises an administrable material storage region 22, which is contained in a three-dimensional sector of the replaceable reservoir or cartridge 20. The hand-held drug applicator 10 has a grounding configuration 90 that directly connect with an external grounding wire configuration 91 through the grounding circuit design 92 and the adjustable grounding circuit design 301. The cumulated electrostatic field generating by the high voltage circuit forms in the vicinity of the spraying nozzles region 31 is conducted away from the spraying nozzles region 31.

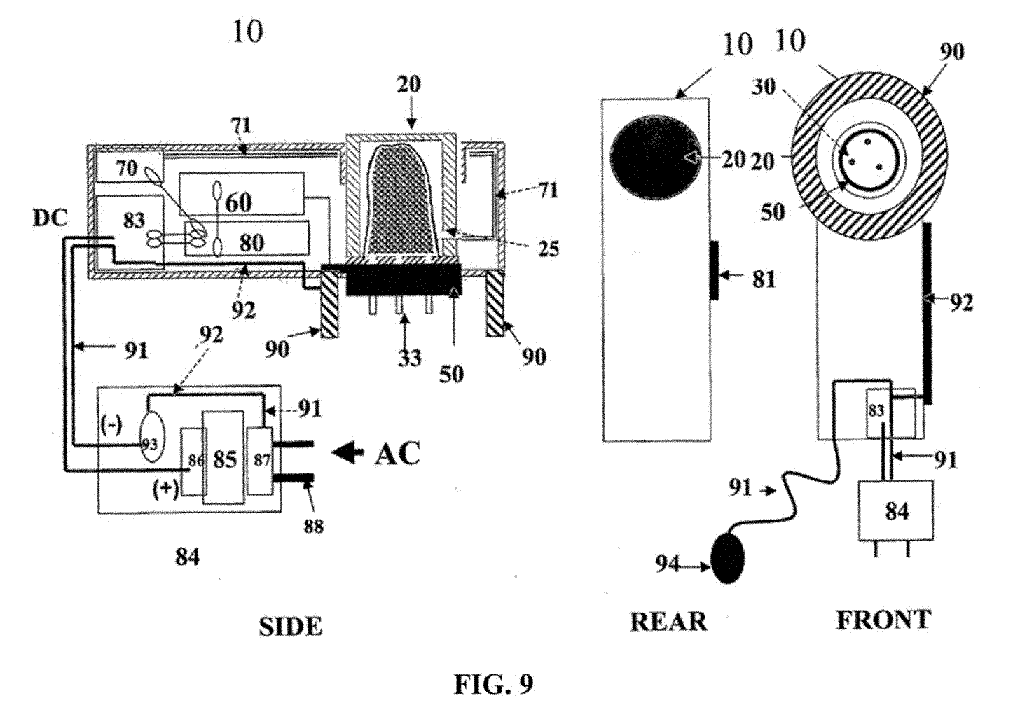

In one alternative embodiment shown in FIG. 9, a complete hand-held drug applicator 10 having replaceable reservoir or cartridge 20, at least one replaceable spraying nozzles region 33, a ring configuration 50, a grounding configuration 90, a special AC to DC adaptor 84, a high voltage generator 60, a power source 82, an external grounding wire configuration 91, control circuit 80, a grounding circuit design 92, a trigger 81, a pumping means 70 and terminals 61 connected to the high voltage generator 60 made in accordance with the first preferred embodiment of the present invention is shown in FIG. 9. The replaceable reservoir or cartridge 20 shown in FIG. 9 comprises a administrable material storage region 22, which is contained in a three-dimensional sector of the replaceable reservoir or cartridge 20. The hand-held drug applicator 10 has a grounding configuration 90 that directly connect with an external grounding wire configuration 91 and a special AC to DC adaptor 84 through the grounding circuit design 92. The cumulated electrostatic field generating by the high voltage circuit forms in the vicinity of the spraying nozzles region 31 is conducted away from the spraying nozzles region 31 and then released into a ground or an AC power source through the special AC to DC adaptor 84.

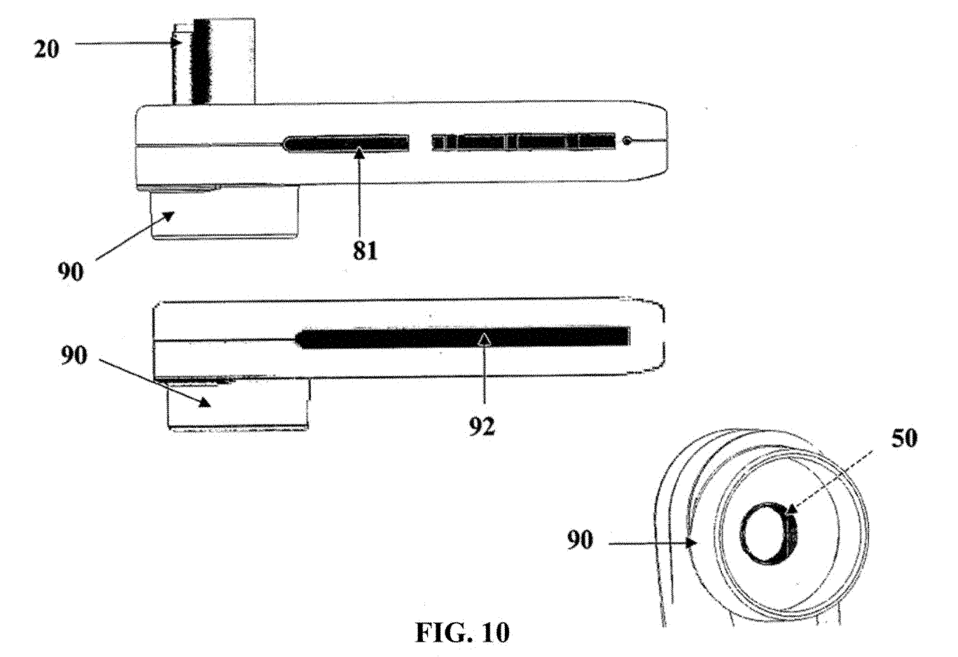

FIG. 10 depicts variants of the fourth preferred embodiment in which the grounding configuration 90 and the grounding circuit design 92 are constructed in a relation to the hand-held drug applicator 10.

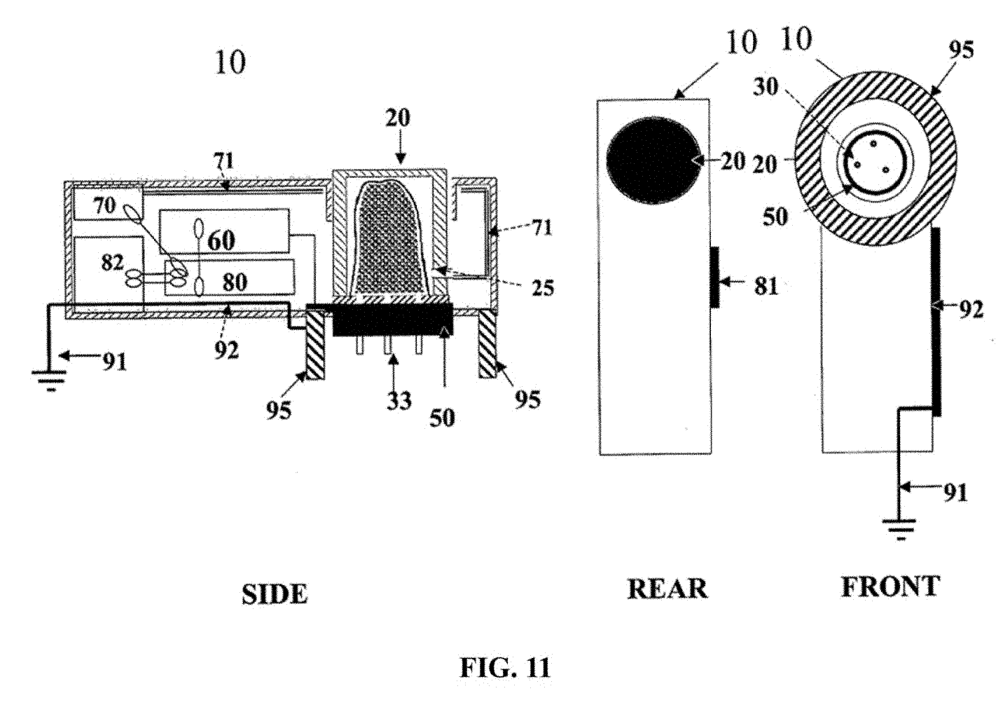

A complete hand-held drug applicator 10 having replaceable reservoir or cartridge 20, at least one replaceable spraying nozzles region 33, a ring configuration 50, a shielding configuration 95, a high voltage generator 60, a power source 82, an external grounding wire configuration 91, control circuit 80, a grounding circuit design 92, a trigger 81, a pumping means 70 and terminals 61 connected to the high voltage generator 60 made in accordance with the fourth preferred embodiment of the present invention is shown in FIG. 11. The replaceable reservoir or cartridge 20 shown in FIG. 11 comprises an administrable material storage region 22, which is contained in a three-dimensional sector of the replaceable reservoir or cartridge 20. The hand-held drug applicator 10 has a shielding configuration 95 that directly connect with an external grounding wire configuration 91 through the grounding circuit design 92. The magnetic wave generating by the high voltage circuit forms in the vicinity of the spraying nozzles region 31 is conducted away from the spraying nozzles region 31.

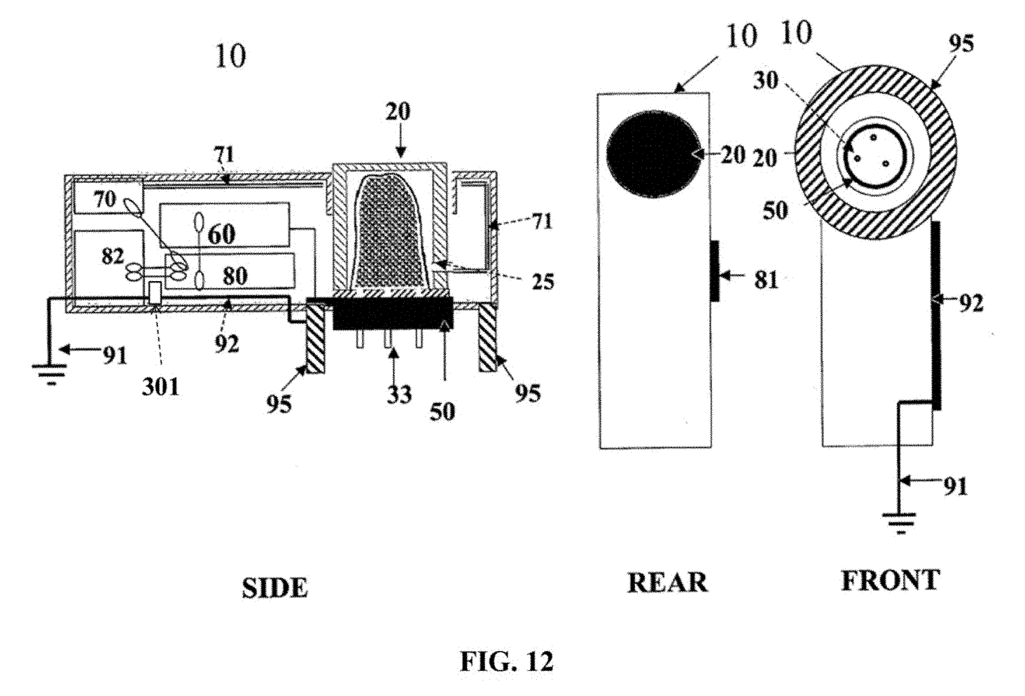

A complete hand-held drug applicator 10 having replaceable reservoir or cartridge 20, at least one replaceable spraying nozzles region 33, a ring configuration 50, a shielding configuration 95, a high voltage generator 60, a power source 82, an external grounding wire configuration 91, control circuit 80, a grounding circuit design 92, a trigger 81, a pumping means 70 and terminals 61 connected to the high voltage generator 60 made in accordance with the fourth preferred embodiment of the present invention is shown in FIG. 12. The replaceable reservoir or cartridge 20 shown in FIG. 12 comprises an administrable material storage region 22, which is contained in a three-dimensional sector of the replaceable reservoir or cartridge 20. The hand-held drug applicator 10 has a shielding configuration 95 that directly connect with an external grounding wire configuration 91 through the grounding circuit design 92 and the adjustable grounding circuit design 301. The magnetic wave generating by the high voltage circuit forms in the vicinity of the spraying nozzles region 31 is conducted away from the spraying nozzles region

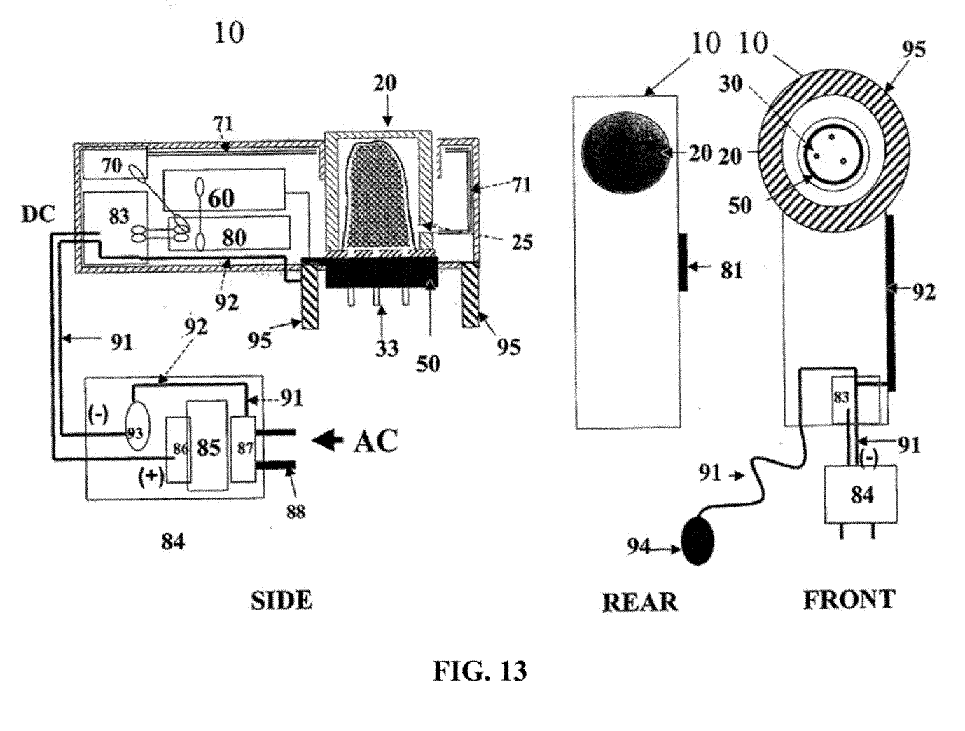

In one alternative embodiment shown in FIG. 13, a complete hand-held drug applicator 10 having replaceable reservoir or cartridge 20, at least one replaceable spraying nozzles region 33, a ring configuration 50, a shielding configuration 95, a special AC to DC adaptor 84, a high voltage generator 60, a power source 82, an external grounding wire configuration 91, control circuit 80, a grounding circuit design 92, a trigger 81, a pumping means 70 and terminals 61 connected to the high voltage generator 60 made in accordance with the fourth preferred embodiment of the present invention is shown in FIG. 13. The replaceable reservoir or cartridge 20 shown in FIG. 13 comprises a administrable material storage region 22, which is contained in a three-dimensional sector of the replaceable reservoir or cartridge 20. The hand-held drug applicator 10 has a shielding configuration 95 that directly connect with an external grounding wire configuration 91 and a special AC to DC adaptor 84 through the grounding circuit design 92. The magnetic wave generating by the high voltage circuit forms in the vicinity of the spraying nozzles region 31 is conducted away from the spraying nozzles region 31 and then released into a ground or an AC power source through the special AC to DC adaptor 84.

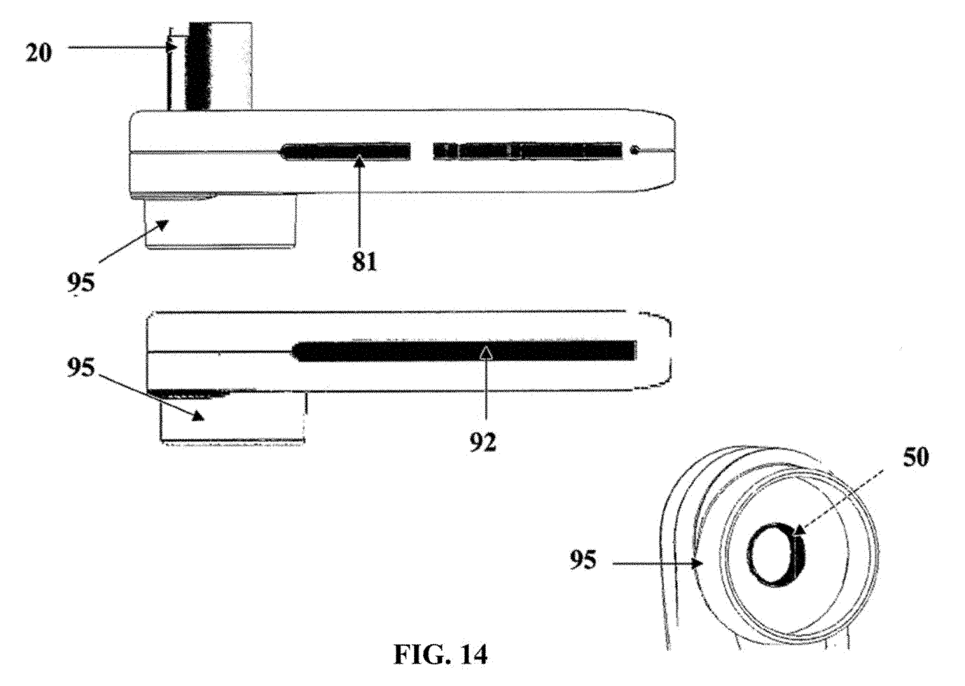

FIG. 14 depicts variants of the fourth preferred embodiment in which the shielding configuration 95 and the grounding circuit design 92 is constructed in a relation to the hand-held drug applicator 10.

1. A method for the contactless administration of a bioactive agent to a tissue, comprising: contactless administration to the tissue with a replaceable reservoirs or cartridge for use with the drug applicator and the drug applicator, the replaceable reservoirs or cartridge loaded with the bioactive agent; and passing an electrostatically discharge through the replaceable reservoirs or cartridge in a direction that eject the bioactive towards the tissue.

2. The method according to claim 1, wherein the replaceable reservoirs or cartridge is for storing at least one electrostatically charged bioactive agent.

3. The method according to claim 1, wherein the bioactive agent is non-charged in nature.

4. The method according to claim 1, wherein the drug applicator comprising special arranged grounding configuration so that, in use, the cumulated electrostatic field generating by the high voltage circuit forms in the vicinity of the administration nozzles region is conducted away from the administration nozzles region.

5. A method for the contactless administration of a bioactive agent to a tissue, comprising: contactless administration to the tissue with a replaceable reservoirs or cartridge for use with the drug applicator and the drug applicator, the replaceable reservoirs or cartridge loaded with the bioactive agent; and passing an electrostatically discharge through the replaceable reservoirs or cartridge in a direction that eject the bioactive towards the tissue. The inconsecutive administration of charged bioactive particle applying to the tissue an electrical gradient, such that the charged bioactive moves into the tissue under the influence of the electrical field.

6. The method according to claim 5, wherein the replaceable reservoirs or cartridge is for storing at least one electrostatically charged bioactive agent.

7. The method according to claim 5, wherein the bioactive agent is non-charged in Nature.

8. The method according to claim 5, wherein the administration of electrical gradient, is generated through the administration of charged bioactive particles or ionic air.

9. The method according to claim 5, wherein the drug applicator comprising special arranged grounding configuration so that, in use, the electrical field generating by the charged bioactive particles or ionic air on the tissue is conducted away in a controllable manner of adjusting the grounding frequency and the output frequency of the high voltage generator.

10. A method for the contactless administration of a bioactive agent to a tissue, comprising: contactless administration to the tissue with a replaceable reservoirs or cartridge for use with the drug applicator and the drug applicator, the replaceable reservoirs or cartridge loaded with the bioactive agent; and passing an electrostatically discharge through the replaceable reservoirs or cartridge in a direction that eject the bioactive towards the tissue. The inconsecutive administration of charged bioactive particle applying to the tissue a high voltage pulses, such that the charged bioactive transport through transiently permeabilized tissue by electroporation.

11. The method according to claim 10, wherein the replaceable reservoirs or cartridge is for storing at least one electrostatically charged bioactive agent.

12. The method according to claim 10, wherein the bioactive agent is non-charged in nature.

13. The method according to claim 10, wherein the administration of high voltage pulses is generated through the inconsecutive administration of charged bioactive particles or ionic air.

14. The method according to claim 10, wherein the administration of high voltage pulses can be adjusted in a fashion of changing the output frequencies of the high voltage generator.

15. The method according to claim 10, wherein the drug applicator comprising special arranged grounding configuration so that, in use, the high voltage generating by the charged bioactive particles or ionic air on the tissue is conducted away in a controllable manner of adjusting the grounding frequency and the output frequency of the high voltage generator.

16. A method for the contactless administration of a bioactive agent to a tissue, comprising: contactless administration to the tissue with a replaceable reservoirs or cartridge for use with the drug applicator and the drug applicator, the replaceable reservoirs or cartridge loaded with the bioactive agent; and passing an electrostatically discharge through the replaceable reservoirs or cartridge in a direction that eject the nanonized bioactive towards the tissue in a manner of nanospray.

17. The method according to claim 16, wherein the replaceable reservoirs or cartridge is for storing at least one electrostatically charged bioactive agent.

18. The method according to claim 16, wherein the bioactive agent is nanonized or non-nanonized in nature.

19. The method according to claim 16, wherein the drug applicator comprising special arranged grounding configuration so that, in use, the cumulated electrostatic field generating by the high voltage circuit forms in the vicinity of the administration nozzles region is conducted away from the administration nozzles region.

20. A replaceable reservoirs or cartridge for use with drug applicator of the type wherein at least one electrostatically administrable material is contained in a reservoir housing, the drug applicator comprising high voltage circuitry arranged so that, in use, the material issuing from the administration nozzles in a direction away from said administration nozzles forms an electrostatically charged spray, said spray having a high voltage and a polarity and wherein an electric field forms in the vicinity of the nozzles during use, the replaceable reservoirs or cartridges comprising:
at least one electrostatically administrable material storage configuration;
at least one replaceable or fixed administration nozzles configuration;
at least one connecting port configuration for conducting the high voltage to the nozzle-ring configuration so that administration from the nozzles is focused when the forward extremity of the nozzle-ring configuration is brought within a predetermine distance from an earthed target to be sprayed.
